(19) United States
(12) Patent Application Publication
(10) Pub. No.: US 2005/0154051 A1
(43) Pub. Date: Jul. 14, 2005

(54) Method for the preparation of escitalopram

(75) Inventors: Haleh Ahmadian, Solrod Strand (DK); Hans Petersen, Vanlose (DK)

Correspondence Address: DARBY & DARBY P.C., P. O. BOX 5257, NEW YORK, NY, 10150-5257, US

(73) Assignee: H. Lundbeck A/S, Copenhagen-Valby (DK)

(21) Appl. No.: 10/498,747

(22) PCT Filed: Dec. 9, 2002
(86) PCT No.: PCT/DK02/00837

Related U.S. Application Data
(60) Provisional application No. 60/340,450, filed on Dec. 14, 2001.

(30) Foreign Application Priority Data
Dec. 14, 2001 (DK) PA 2001 01881

(51) Int. Cl. A61K 31/343; C07D 307/87

(52) U.S. Cl. 514/469; 549/467

(57) ABSTRACT

The invention relates to a method for the preparation of escitalopram by cyanation of optically active intermediates of the formulas (III) and (II) below, and the preparation of such intermediates by optical resolution.

The present invention relates to a novel method for the preparation of escitalopram (the S-enantiomer of citalopram) from the S-enantiomer of a citalopram derivative and to the preparation of said S-enantiomer of a citalopram derivative.

### BACKGROUND OF THE INVENTION

Citalopram is a well-known antidepressant drug that has now been on the market for some years and has the following Formula:

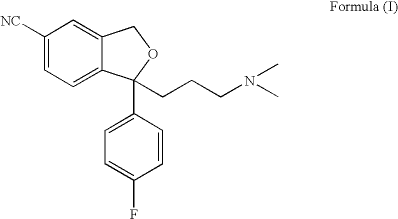

It is a selective, centrally acting serotonin (5-hydroxytryptamine; 5-HT) reuptake inhibitor, accordingly having antidepressant activities.

Citalopram was first disclosed in DE 2,657,013, corresponding to U.S. Pat. No. 4,136,193. This patent publication i.a. outlines a process for the preparation of citalopram from the corresponding 5-bromo-derivative by reaction with cuprous cyanide in a suitable solvent. Further processes for the preparation of citalopram by exchange of 5-halogen or 5-CF₃—(CF₂)ₙ—SO₂—O—, n being 0-8, with cyano are disclosed in WO 00/11926 and WO 00/13648.

U.S. Pat. No 4,943,590 corresponding to EP-B1-347 066 describes two processes for the preparation of escitalopram.

Both processes use the racemic diol having the formula 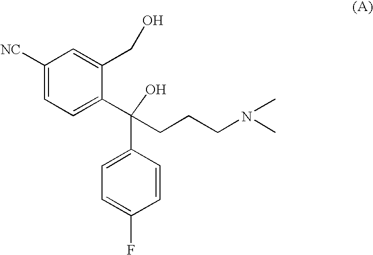
as starting material. According to the first process, the diol of formula (A) is reacted with one of the enantiomers of an optically active acid derivative, such as (+) or (−)-α-methoxy-α-trifluoromethyl-phenylacetyl chloride to form a mixture of diastereomeric esters, which are separated by HPLC or by fractional crystallization, whereupon the ester with the right stereochemistry is enantioselectively converted into escitalopram. According to the second process, the diol of formula (A) is separated into the enantiomers by stereoselective crystallisation of a salt with one of the enantiomers of an optically active acid, such as (+)-di-p-toluoyltartaric acid, whereupon the S-enantiomer of the diol of the formula (A) is enantioselectively converted to escitalopram.

Escitalopram is now marketed as an antidepressant Hence, there is a desire for an improved method for preparation of escitalopram.

### THE PRESENT INVENTION

Accordingly the present invention relates to a novel process for the preparation of escitalopram having the formula 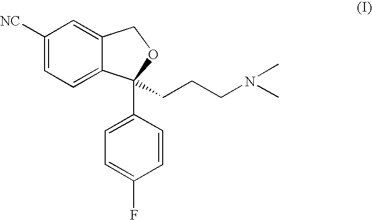
comprising
a) optical resolution of the racemic compound having the formula 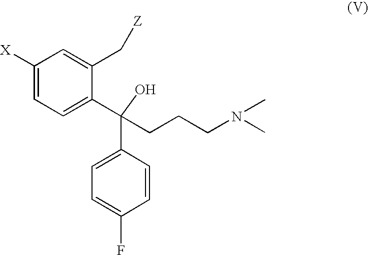
   wherein X is as defined above and Z is OH or a leaving group, by fractional crystallisation of a diastereomeric salt thereof, or by formation and separation of diastereomeric esters thereof optionally followed by hydrolysis of the correct diastereomeric ester, to form a compound of formula 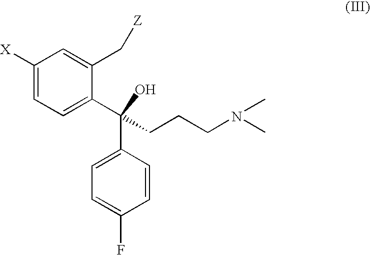
   wherein X is as defined above and Z is OH or a leaving group, and when Z is OH conversion of Z to a leaving group followed by ring closure of the compound of formula (III) to form a compound of formula (II) 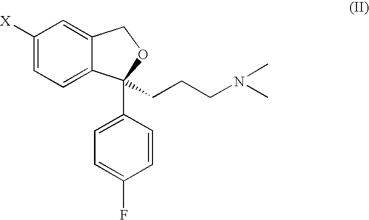
   wherein X is halogen or any other group that may be converted to a cyano group, or by
b) optical resolution of the racemic compound of formula 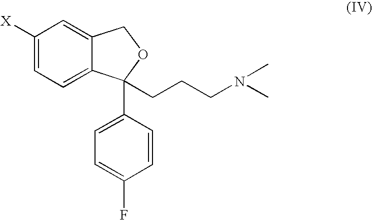
   wherein X is as defined above, by fractional crystallisation of a diastereomeric salt thereof, to form a compound of formula (II) 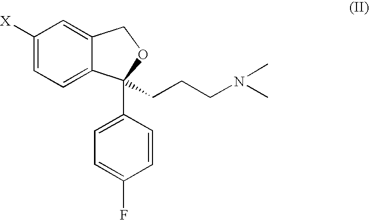
   wherein X is halogen or any other group that may be converted to a cyano group;
and thereafter conversion of the group X in the compound of formula (II) to a cyano group and isolation of escitalopram in the form of the base or a pharmaceutically acceptable salt thereof.

### DETAILED DESCRIPTION OF THE INVENTION

The racemic compound of formula (IV) and the racemic compound of formula (V) may be resolved by fractional crystallization of diastereomeric salts thereof. Suitable optically active acids for the formation of diastereomeric salts include: tartaric acids, such as dibenzoyltartaric acid, di-(p-toluoyl)tartaric acid and o-nitrobenzoyl tartaric acid, lactic acid, bisnapthylphosphoric acid, camphorsulfonic acids, such as 8-camphorsulphonic acid and 10-camphorsulphonic acid, mandelic acid, malic acid and 2-phenoxypropionic acid and derivatives thereof.

The fractional crystallisation and isolation of a diastereomeric salt is suitably carried out by treatment of the free base of a compound of formula (IV) or (V) with one of the enantiomers of an optically active acid in an appropriate solvent which may either be a polar solvent, such as water, alcohols containing 1-8 carbon atoms, acetonitrile and acetone or a polar solvents such as, ethers containing 1-8 carbon atoms and alkanes containing 1-8 carbon atoms. As a result, two diastereomeric salts may be formed, which differ in their stability and solubility properties. The disastereomeric salts may be separated by fractional crystallisation.

The compound of formula (II) and (III) may be liberated from their respective diastereomeric salts by treatment with a base.

The compounds of formula V, wherein Z is OH, may also be resolved by formation and separation of diastereomeric ester thereof. According to this embodiment of the invention, the compound of formula V, wherein Z is OH, is reacted with one of the enantiomers of an optically active acid derivative, such as an acid chloride, anhydride or a labile ester, to form diastereometic esters. The formation of the ester is suitably performed in an inert organic solvent such as toluene, dichloromethane, tetrahydrofuran and acetonitrile. A base, such as triethylamine, N,N-dimethylaniline, pyridine or diisopropylethylamine may be added to neutralise liberated H⁺. In principle, acid derivatives for the formation of diastereomeric esters may be derived from any chiral acid. Suitable chiral acids include tartaric acids, camphanic acids, N-substituted cinnamoylproline derivatives, campher sulfonic acids (campher-10-sulfonic acid, campher-8-sulfonic acid, 3-bromo-campher-10-sulfonic acid, 3-bromo-campher-8-sulfonic acid), optically active amino acids and derivatives thereof (phenylglycine, 4-hydroxyphenylglycine, m-tyrosine, 3,4-dihydroxyalanine, 3,5-diiodothyrosine, N-trifluoroacetylproline), 2-aryl-alkanoic acids (2-phenylpropionic acid, 2-(6-methoxynaphth-2-yl)-propionic acid), menthyl-3-yl-oxyacetic acid, cis and trans chrysanthemic acid, α-methoxy-α-trifluoromethylphenylacetic acid, 2-isopropyl-4′-chlorophenyl acetic acid, mandelic acids, N-benzoyl-cis-2-aminocyclohexanecarboxylic acid, 2-4-chlorophenyl)isovaleric acid, permethrinic acids and 1,1′-binapthyl-2,2′-diylphosphate and derivatives of such acids.

The diastereomeric esters formed may be separated by chromatography, including in particular liquid chromatography or by fractional crystallisation of a salt thereof. The diastereomeric ester of formula (III) with the correct configuration may be treated directly with a strong base in an inert organic solvent to form the compound of formula (II).

The following optically active acid derivatives have been found very useful for the formation of diastereomeric esters: (S)-2-(6-methoxynaphth-2-yl)-propionyl chloride, (S)-2-(4-isobutylphenyl)propionyl chloride, (S)—O-acetylmandeloyl chloride, (S)-benzyloxycarbonylprolyl chloride, (S)-2-phenylbutyryl chloride, ((S)-α-methoxy-phenylacetyl chloride and (S)—N-acetyl-alanine. The diastereomeric esters formed with these acid derivatives may be separated by chromatography and after isolation of the correct distereomer, treatment with a base in an inert organic solvent as described below leads directly to formation of a compound of formula (II).

Alternatively, if the ester formed is not a good leaving group, the diastereomeric ester of formula (III) may be treated with a base, such as NaOH, KOH, NH₃, Ba(OH)₂ or LiOH in a mixture of water and an organic solvent such as toluene, THF or diethylether or with NH₃, NaH, KOC(CH₃)₃, triethylamine or diisopropylethylamine in an inert organic solvent, such as toluene, tetrahydrofuran, dimethoxyethane, dioxane or acetonitrile, yielding the compound of formula (III) wherein Z is OH.

The group Z in the compound of formula (III) wherein Z is OH is then converted to a suitable leaving group. A suitable leaving group is any group which upon treatment of the compound of formula (III) carrying the group with a base in an inert organic solvent, as described below, leads to ringclosure of the compound of formula (III). Suitable leaving groups are sulfonate esters or a halides. The sulfonate esters are formed by reaction with sulfonyl halides, such as methanesulfonyl chloride and p-toluenesulfonyl chloride. The halides are obtained by reaction with halogenating agents such as thionyl chloride or phosphorus tribromide.

Ring closure of the compounds of formula (III) wherein Z is a leaving group, for example sulfonate ester or halogen, to form a compound of formula (II), may thereafter be carried out by treatment with a base such as KOC(CH₃)₃ and other alkoxides, NaH and other hydrides, triethylamine, ethyldiisopropylamine or pyridine in an inert organic solvent, such as tetrahydrofuran, toluene, DMSO, DMF, t-butyl methyl ether, dimethoxyethane, dimethoxymethane, dioxane, acetonitrile and dichloromethane.

This process has already been described in U.S. Pat. No. 4,943,590.

As mentioned above, X may be halogen, preferably chloro or bromo, or any other compound which may be converted to a cyano group.

Such groups, X, may be selected from the groups of formula CF₃—(CF₂)ₙ—SO₂—O—, wherein n is 0-8, —OH, —CHO, —CH₂OH, —CH₂NH₂, —CH₂NO₂, —CH₂Cl, —CH₂Br, —CH₃, —NHR¹, —COOR², —CONR²R³ wherein R¹ is hydrogen or alkylcarbonyl and R² and R³ are selected from hydrogen, optionally substituted alkyl, aralkyl or aryl and,
a group of formula 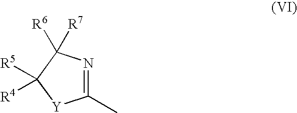
   wherein Y is O or S;
R⁴-R⁵ are each independently selected from hydrogen and C₁₋₆ alkyl or R⁴ and R⁵ together form a C₂₋₅ alkylene chain thereby forming a spiro ring; R⁶ is selected from hydrogen and C₁₋₆ alkyl, R⁷ is selected from hydrogen, C₁₋₆ alkyl, a carboxy group or a precursor group therefore, or R⁶ and R⁷ together form a C₂₋₅ alkylene chain thereby forming a spiro ring.

When X is halogen, in particular bromo or chloro, conversion of the compound of formula (II) to form escitalopram may be carried out as described in U.S. Pat. No. 4,136,193, WO 00/13648, WO 00/11926 and WO 01/02383.

According to U.S. Pat. No. 4,136,193 conversion of the 5-bromo group in a compound corresponding to the compound of formula (II) to a cyano group, is carried out by reaction with CuCN. WO 00/13648 and WO 00/11926 describe the conversion of a 5-halogen or a triflate group in a compound corresponding to the compound of formula (II) to a cyano group by cyanation with a cyanide source in presence of a Pd or Ni catalyst.

The cyanide source used according to the catalysed cyanide exchange reaction may be any useful source. Preferred sources are KCN, NaCN or (R′)₄NCN, where (R′)₄ indicates four groups which may be the same or different and are selected from hydrogen and straight chain or branched C₁₋₆ alkyl.

The cyanide source is used in a stoichiometric amount or in excess, preferably 1-2 equivalents are used pr. equivalent starting material. (R′)₄N⁺ may conveniently be (Bu)₄N⁺. The cyanide source is preferably NaCN or KCN or Zn(CN)₂.

The palladium catalyst may be any suitable Pd(O) or Pd(II) containing catalyst, such as Pd(PPh₃)₄, Pd₂(dba)₃, Pd(PPh)₂Cl₂, etc. The Pd catalyst is conveniently used in an amount of 1-10, preferably 2-6, most preferably about 4-5 mol %.

In one embodiment of the invention, the reaction is carried out in the presence of a catalytic amount of Cu⁺ or Zn²⁺.

Catalytic amounts of Cu⁺ and Zn²⁺, respectively, mean substoichiometric amounts such as 0.1-5, preferably 1-3 mol %. Conveniently, about ½ eq. is used per eq. Pd. Any convenient source of Cu⁺ and Zn⁺⁺ may be used. Cu⁺ is preferably used in the form of CuI, and Zn²⁺ is conveniently used as the Zn(CN)₂ salt.

In a preferred embodiment, cyanation is carried out by reaction with ZnCN₂ in the presence of a Palladium catalyst, preferably Pd(PPh₃)₄ (tetralds(triphenylphosphine)palladium).

The nickel catalyst may be any suitable Ni(0) or Ni(II) containing complex which acts as a catalyst, such as Ni(PPh₃)₃, (σ-aryl)-Ni(PPh₃)₂Cl, etc. The nickel catalysts and their preparation are described in WO 96/11906, EP-A-613720 and EP-A-384392.

In a particularly preferred embodiment, the nickel(0) complex is prepared in situ before the cyanation reaction by reduction of a nickel(II) precursor such as NiCl₂ or NiBr₂ by a metal, such as zinc, magnesium or manganese in the presence of excess of complex ligands, preferably triphenylphosphin.

The Ni-catalyst is conveniently used in an amount of 0.5-10, preferably 2-6, most preferably about 4-5 mol %.

In one embodiment of the invention, the reaction is carried out in the presence of a catalytic amount of Cu⁺ or Zn²⁺.

Catalytic amounts of Cu⁺ and Zn²⁺, respectively, mean substoichiometric amounts such as 0.1-5, preferably 1-3%. Any convenient source of Cu⁺ and Zn²⁺ may be used. Cu⁺ is preferably used in the form of CuI, and Zn²⁺ is conveniently used as the Zn(CN)₂ salt or formed in situ by reduction of a nickel (II) compound using zinc.

The cyanation reaction may be performed neat or in any convenient solvent, such solvent includes DMF, NMP, acetonitril, propionitrile, THF and ethylacetate.

The cyanide exchange reaction may also be performed in an ionic liquid of the general formula (R″)₄N⁺, Y⁻, wherein R″ are alkyl-groups or two of the R″ groups together form a ring and Y⁻ is the counterion. In one embodiment of the invention, the ionic liquid is represented by the formula

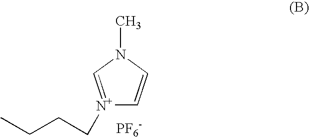

In still another alternative, the cyanide exchange reaction is conducted with a polar solvents such as benzene, xylene or mesitylene and under the influence of microwaves by using i.e. Synthewave 1000™ by Prolabo

The temperature ranges are dependent upon the reaction type. If no catalyst is present, preferred temperatures are in the range of 100-200 ° C. However, when the reaction is conducted under the influence of microwaves, the temperature in the reaction mixture may raise to above 300° C. More preferred temperature ranges are between 120-170° C. The most preferred range is 145-155° C.

If a catalyst is present, the preferred temperature range is between 0 and 100° C. More preferred are temperature ranges of 40-90° C. Most preferred temperature ranges are between 60-90° C.

Other reaction conditions, solvents, etc. are conventional conditions for such reactions and may easily be determined by a person skilled in the art.

Other processes for the conversion of a compound of formula (II) wherein X is bromo to the corresponding 5-cyano derivative involve reaction of 5-bromocitaloprarm with magnesium to form a Grignard reagent, followed by reaction with a formamide to form an aldehyde. The aldehyde is converted to an oxime or a hydrazone which is converted to a cyano group by dehydration and oxidation, respectively.

Alternatively, compound of formula (II) wherein X is bromo is reacted with magnesium to form a Grignard reagent, followed by reaction with a compound containing a CN group bound to a leaving group.

A detailed description of the above two procedures may be found in WO 01/02383.

Compounds of formula (II), wherein the group X is CF₃—(CF₂)ₙ—SO₂—O—, wherein n is 0-8, may be converted to escitalopram by methods analogous to those described in WO 00/13648.

Compounds of formula (II), wherein the group X is —CHO, may be converted to escitalopram by methods analogous to those described in WO 99/00210.

Compounds of formula (II), wherein the group X is NHR¹, wherein R¹ is hydrogen or alkylcarbonyl, may be converted by to escitalopram methods analogous to those described in WO 98/19512.

Compounds of formula (II), wherein the group X is —CONR²R³, wherein R² and R³ are selected from hydrogen and optionally substituted alkyl, aralkyl or aryl may be converted to escitalopram by methods analogous to those described in WO 98/00081 and WO 98/19511. Compounds of formula (II), wherein the group X is a group of formula (VI) may be converted to escitalopram by methods analogous to those described in WO 00/23431.

Compounds of formula (II), wherein X is OH, —CH₂OH, —CH₂NH₂, —CH₂NO₂, —CH₂Cl, —CH₂Br, —CH₃ or any of the groups above, may be converted to escitalopram by methods analogous to those described in WO 01/168632.

Starting materials of formula (IV) or (V) may be prepared according to the above mentioned patents and patent applications or by analogous methods.

Methods

Formation of Diastereomeric Esters:

General Procedure:

A mixture of an enantiomerically pure acid (S-enantiomer) (1.3 eqv.) and thionyl chloride (10 eqv) and a few drops of dimethylformamide in toluene (50 mL) is heated to reflux for ½ h. after cooling to room temperature, evaporation and re-evaporation from toluene, the residue is dissolved in dry THF (10% w/v solution) and added to a solution of 1-(4-bromo-2-hydroxymethyl-phenyl)-4-dimethylamino-1-(4′-fluorophenyl)-butan-1-ol., (1 eqv.) and triethylamine (1.5 to 2 eqv.) and dimethylaminopyridine (DMAP) (catalytic amount) in THF (50 mL). The resulting mixture is stirred at room temperature overnight. After filteration and evaporation, silica gel chromatography (EtOAc; n-heptane; triethylamine16: 8: 1) a mixture of two diastereomeric esters may be obtained as a residue.

Separation of the Diastereomers:

General Procedure:

A column with the dimensions 4.6×250 mm packed with Daice® AD (5 μm particle size) is used as the stationary phase. The mobile phase that is used is carbon dioxide and a modifier in a ratio of 90:10. The modifier may be methanol with diethylamine (0.5%) and trifluoroacetic acid (0.5%). The operation conditions is as follows:
Temperature: room temperature
Flow rate: 2 ml/min
Detection: UV 210 and 254 nm

Pressure: 20 MPa

The identification of the (S,S) and (S,R) diastereomers is based on comparison with the retention times of the corresponding esters synthesised from (S)-1-(4-bromo-2-hydroxymethyl-phenyl)-4-dimethylamino-1-(4-fluorophenyl)-butan-1-ol and the (S)-enantiomers of acid chlorides.

Ring Closure of the (S,S)-enantiomer of the Esters to make Escitalopram:

General Procedure:

NaH (1.1 eqv., 60% dispersion in mineral oil) is added to a solution of the (S,S)-enantiomer of the ester in DMF (5% w/v solution) at room temperature. The resulting mixture is stirred for 1 h, then poured into saturated ammonium chloride solution and extracted with diethyl ether three times. The combined organic phases are extracted twice with 1 M HCl solution. The aqueous phase is basified with konc. NaOH and extracted twice with diethyl ether. The organic phases are dried (MgSO₄), filtered and evaporated to afford crude (S)-Br-citalopram.

1. A method for the preparation of escitalopram having the formula 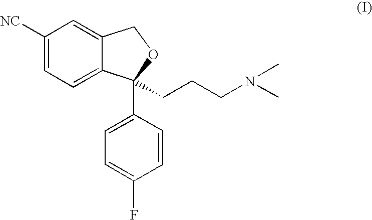
comprising
a) optical resolution of the racemic compound having the formula 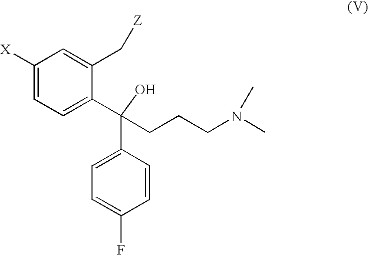
wherein X is halogen or any other group that may be converted to a cyano group, and Z is OH or a leaving group by fractional crystallisation of a diastereomeric salt thereof, or by formation and separation of diastereomeric esters thereof optionally followed by hydrolysis of the correct diastereomeric ester to form a compound of formula 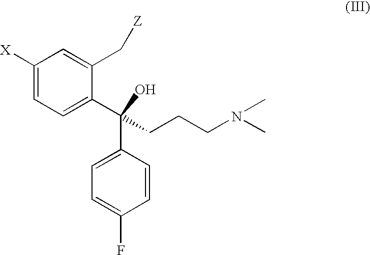
wherein X is as defined above and Z is OH or a leaving group, and when Z is OH conversion of Z to a leaving group, followed by ring closure of the compound of formula (III) to form a compound of 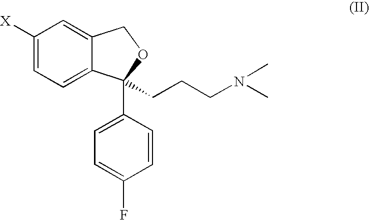
wherein X is halogen or any other group that may be converted to a cyano group; or
b) optical resolution of the racemic compound of formula 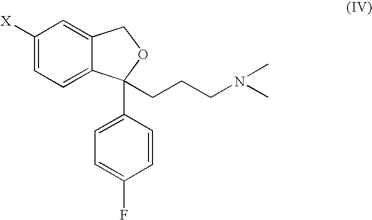
wherein X is as defined above, by fractional crystallisation of a diastereomeric salt thereof to form a 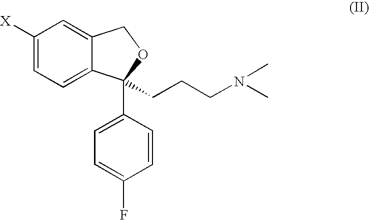
wherein X is halogen or any other group that may be converted to a cyano group;
followed by conversion of the group X in the compound of formula (II) to a cyano group and thereafter isolation of escitalopram in the form of the base or as a pharmaceutically acceptable salt thereof.

2. The method according to claim 1, wherein the racemic compound of formula (IV) is resolved by fractional crystallisation of a diastereomeric salt formed with one of the enantiomers of an optically active acid optionally followed by treatment with a base to form the free base of the compound of formula (II).

3. The method according to claim 1, wherein the racemic compound of formula (V) is resolved by reaction with one of the enantiomers of an optically active acid derivative followed by separation of the diastereomeric esters formed by chromatography or fractional crystallisation of a salt thereof, followed by separation of the correct diastereomeric ester to form a compound of formula (II), or followed by treatment of the correct diastereomeric ester with a base in presence of water to form a compound of formula (III) wherein Z is OH, thereafter conversion of the group Z to a leaving group and then ring closure to form a compound of formula (II).

4. The method according to claim 1, wherein the racemic compound of formula (V) is resolved by fractional crystallisation of a diastereomeric salt formed with one of the enantiomers of an optically active acid, optionally followed by treatment with a base to form the free base of the compound of formula (III) and where Z is not a leaving group, conversion of Z to a leaving group and then ring closure to form a compound of formula (II).

5. The method according to claim 1, wherein the group X is bromo.

6. The method of claim 1, wherein the optically active acid used for the formation of a diastereomeric salt is an enantiomer of tartaric acid, lactic acid, bisnapthylphosphoric acid, camphorsulfonic acids, mandelic acid, malic acid or 2-phenoxypropionic acid or a derivative of any of these acids.

7. The method according to claims 3, wherein the optically active acid used for the formation of diastereomeric esters is an enantiomer of α-methoxy-α-trifluoromethyl-phenylacetic acid, a mandelic acid acids, a tartaric acid, 2-aryl-alkanoic acids, an optically active amino acid, a camphanic acid or a derivative of any of these acids.

8. The method according to claim 7 wherein the optically active acid derivative used for the formation of diastereomeric esters is (S)-2-(6-methoxynaphth-2-yl)-propionyl chloride, (S)-2-(4-isobutylphenyl)propionyl chloride, (S)—O-acetylmandeloyl chloride, (S)-benzyloxycarbonylprolyl chloride, (S)-2-phenylbutyryl chloride, (S)-α-methoxy-phenylacetyl chloride or (S)—N-acetyl-alanine.

9. The method according to claim 1, wherein a compound of formula (II) wherein X is halogen, in particular bromo is formed and thereafter converted to escitalopram by reaction of a compound of formula (II) with CuCN followed by purification and isolation of escitalopram or a pharmaceutically acceptable salt thereof.

10. The method according to claim 1, wherein a compound of formula (II) wherein X is halogen, in particular bromo, or CF₃—(CF₂)ₙ—SO₂—O—, wherein n is 0-8, is formed and thereafter converted to escitalopram by reaction of the compound of formula (II) with cyanide source in presence of a palladium catalyst optionally followed by purification and isolation of escitalopram or a pharmaceutically acceptable salt thereof.

11. The method according to claim 1, wherein a compound of formula (II) wherein X is halogen, in particular chloro, is formed and thereafter converted to escitalopram by reaction of a compound of formula (II) with cyanide source in presence of a nickel catalyst optionally followed by purification and isolation of escitalopram or a pharmaceutically acceptable salt thereof.

12. The method according to claim 2, wherein the group X is bromo.

13. The method according to claim 3, wherein the group X is bromo.

14. The method according to claim 4, wherein the group X is bromo.

15. The method of claim 2, wherein the optically active acid used for the formation of a diastereomeric salt is an enantiomer of tartaric acid, lactic acid, bisnapthylphosphoric acid, camphorsulfonic acids, mandelic acid, malic acid or 2-phenoxypropionic acid or a derivative of any of these acids.

16. The method of claim 4, wherein the optically active acid used for the formation of a diastereomeric salt is an enantiomer of tartaric acid, lactic acid, bisnapthylphosphoric acid, camphorsulfonic acids, mandelic acid, malic acid or 2-phenoxypropionic acid or a derivative of any of these acids.

17. The method of claim 5, wherein the optically active acid used for the formation of a diastereomeric salt is an enantiomer of tartaric acid, lactic acid, bisnapthylphosphoric acid, camphorsulfonic acids, mandelic acid, malic acid or 2-phenoxypropionic acid or a derivative of any of these acids.

18. The method of claim 12, wherein the optically active acid used for the formation of a diastereomeric salt is an enantiomer of tartaric acid, lactic acid, bisnapthylphosphoric acid, camphorsulfonic acids, mandelic acid, malic acid or 2-phenoxypropionic acid or a derivative of any of these acids.

19. The method of claim 14, wherein the optically active acid used for the formation of a diastereomeric salt is an enantiomer of tartaric acid, lactic acid, bisnapthylphosphoric acid, camphorsulfonic acids, mandelic acid, malic acid or 2-phenoxypropionic acid or a derivative of any of these acids.
